(19) United States
(12) Patent Application Publication
(10) Pub. No.: US 2011/0301411 A1
(43) Pub. Date: Dec. 8, 2011

(54) FLUIDIC ENDOSCOPE TIP LOCATOR

(76) Inventor: JAMES K. BRANNON, LEAWOOD, KS (US)

(21) Appl. No.: 12/961,487

(22) Filed: Dec. 6, 2010

Related U.S. Application Data
(60) Provisional application No. 61/266,908, filed on Dec. 4, 2009.

(51) Int. Cl. A61B 1/018; A61B 1/12

(52) U.S. Cl. 600/104

(57) ABSTRACT

The present invention provides an improved tip position determining assembly for visually and fluidically determining the entry of an instrument tip into an internal surgical site, the assembly comprising an endoscopic portal having a chamber housing a membrane having a plurality of slit openings associated with a slit for sealingly receiving a trephine and an irrigant and vacuum source connectably secured to said assembly for observing an instrument tip entering a surgical site.

### CROSS-REFERENCE TO RELATED APPLICATION

This application claims the benefit of the prior filed U.S. provisional application No. 61/266,908 filed Dec. 4, 2009 which is incorporated herein by reference.

### FIELD OF THE INVENTION

The present invention is broadly directed to improvements in instruments for endoscopic surgery and, more particularly, for detecting the entry of a tip of an endoscopic instrument assembly within a surgical site within a patient by fluidic means.

### BACKGROUND OF THE INVENTION

Modern surgery tends toward minimally invasive techniques whenever possible. Although often more complicated in some ways for the surgeon, minimally invasive techniques result in less trauma to the patient and less scarring because of much smaller incisions thereby promoting faster healing and reducing possibilities for infections. In general, minimally invasive surgeries involve making one or more small incisions at appropriate locations and inserting tubular devices through the incisions to the surgical site. The tubular devices may be referred to as endoscopes, arthroscopes, and the like and typically have optical fiber based optical viewing apparatus and light sources, surgical instruments, lumens for exchanging fluids with the surgical site, or combinations thereof extending therethrough. In some circumstances it is more appropriate to separate the light source and viewing scope from specifically surgical instruments, thus requiring two incisions and endoscopes. This technique is sometimes referred to as triangulation. In other instances, external types of imaging techniques are used for locating endoscopic instruments, such as fluoroscopes, computed tomography, magnetic resonance imaging, or the like.

Endoscopic instruments are configured in a number of different ways depending on their intended purpose. There are rigid endoscopes and flexible endoscopes. Some are simply tubes or portal instruments which provide access to a surgical site for instruments which are passed through the scopes or for the exchange of fluids to and from the surgical site. Viewing scopes, including light sources, may be used for viewing a surgical site for diagnostic purposes or to view surgical operations occurring through the same scope or a different scope. Surgical operations may include cutting, shaving, debriding, cauterizing, or the like as well as grasping tissues or parts of organs, such as with forceps.

In the use of endoscopic instruments, it is often necessary for the surgeon to locate the surgical site indirectly, that is, without a direct view of the site initially. This is especially true with regard to arthroscopic surgery. It has often been necessary to employ radiopaque endoscopic instruments and a radiant imaging technique, such as fluoroscopy or computed tomography to extend an endoscopic instrument from an external incision to the surgical site. It is desirable to limit irradiation of the patient if alternative procedures can be used to achieve the same objectives.

In some types of endoscopic surgery, a portal instrument or scope is used to maintain a pathway from an external incision to a surgical site, such as a hip joint. The portal scope is of such a diameter that incorrect placement of the distal tip within the joint capsule could injure the femoral head or other organs or tissues of other types of endoscopic surgical sites. Therefore, accurate placement is very desirable. In some situations, it is desirable for the portal scope to be transparent to enable viewing through a portal cannula thereof with a viewing scope to observe the environment of the surgical site. However, such a portal scope with a cannula formed of a transparent plastic would be radiotransparent and not easily imaged with a fluoroscope.

### SUMMARY OF THE INVENTION

The present invention provides improvements in endoscopic diagnosis and surgery by providing a method of locating the entry of the tip of an endoscopic instrument into a surgical site by a non-radiant means. In particular, the present invention employs fluid means for an indication of entry of the endoscopic tip at the surgical sight.

An embodiment of the invention employs a transparent endoscopic portal instrument or scope having an enlarged membrane chamber communicating with an elongated portal cannula terminating distally in a portal cannula tip. The portal cannula tip may be conically tapered and cut off at an angle to the longitudinal axis of the portal cannula. The membrane chamber will remain external to the patient, while the cannula will be extended through an incision toward the surgical site. A side port communicates with the membrane chamber. A rear port communicates with the membrane chamber and is aligned with the portal cannula, but is separated from the membrane chamber by a portal membrane or diaphragm. The membrane has a slit cut therethrough which is aligned with the portal cannula and which normally closed. An embodiment of the portal scope has a crossed pair of slits which intersect at substantially a right angle. The portal scope is employed to establish and maintain a pathway from an external incision in the patient to the surgical site which may, for example, be a knee joint, a hip joint, or the like. The portal scope enables other endoscopic instruments to be passed therethrough to the surgical site, as well as the passage of fluids to and from the surgical site. The portal scope is formed of a transparent plastic which enables viewing through portions thereof, especially the cannula and tip.

The embodiment of the invention includes a trephine or trephine type of instrument including a trephine body having an internal trephine chamber and an elongated trephine cannula extending therefrom. The trephine cannula terminates distally in a trephine tip which may be conically tapered and cut off at an angle. The trephine includes at least one port, such as a rear port communicating with the trephine chamber and aligned with the trephine cannula. The trephine body may also include a side port communicating with the trephine chamber. The trephine instrument need not be transparent and may be constructed from any suitable material, such as a stainless steel, a polymer, or the like.

The trephine cannula has an outer diameter which is less than an inner diameter of the portal cannula. This enables the trephine cannula to be inserted through the rear port of the portal scope, the membrane slits, and through the portal cannula, leaving an annular passage between the outer surface of the trephine cannula and the inner surface of the portal cannula. The trephine cannula needs to be somewhat longer than the entire length of the portal scope.

In an embodiment of the present invention the trephine instrument is used in combination with the portal scope to fluidically determine entry of the portal scope tip into a surgical site. The trephine instrument is inserted entirely through the portal scope with the trephine cannula passing through the rear port and membrane slits of the portal scope. A vacuum source is connected to the side port of the portal scope, and a pressurized fluid source is connected to the trephine instrument. If no side port is available on the trephine instrument, the fluid source can be connected to the rear port. If a side port is present, the fluid source can be connected to either the side port or the rear port, with the other port plugged. Neither the vacuum source nor the fluid source needs to be strongly pressurized. The fluid source provides an irrigant such as water, a saline solution, or other solution of a type which would be used to irrigate a surgical site. The trephine instrument is preferably inserted as far as possible through the portal scope, without closing the rear port.

In an exemplary application, the tip of the trephine cannula is telescoped over a guide wire which has been previously emplaced in the surgical site, as by use of a fluoroscope. The tip of the portal scope, with the trephine tip axially extending theralong, is inserted through an external incision and advanced carefully toward the surgical site along the guide wire, with the surgeon observing the transparent portal scope. Fluid from the fluid source exits the trephine tip, while the negative pressures is presented through an annular cannula gap between the trephine cannula and the portal cannula. Depending on the pressure differential between the tip of the portal scope and the rear port, the negative pressure may be relieved somewhat by air passing through the membrane slits with the trephine cannula passing therethrough, based upon pressure differentials. Small amounts of fluid exiting the trephine tip may be drawn into the cannula gap, for example, if there is a blockage at the portal tip by intervening tissues encountered between the incision and the surgical site. When the trephine tip enters the surgical site, such as a joint capsule, the fluid exiting therefrom fills the site. As the portal tip enters the surgical site, blockage of the portal cannula tip is reduced and the fluid within the surgical site is drawn therein, enabling a substantially increased flow of the fluid into the portal tip and back through the portal cannula toward the side port. Such increased flow is visually observable by the surgeon and indicates that the portal tip has entered the surgical site. At this point, further insertion of the portal tip into the surgical site is halted, and the trephine instrument can be withdrawn from the portal scope. Other instruments can now be extended through the portal scope for diagnosis and surgical treatment as indicated by the conditions observed.

A removable outflow adapter may be rearwardly associated with the trephine, the removable outflow adapter having a main passage extending between a terminus towards a male port and a radial passage extending radially outward from the main passage. A female connector associated with the proximal rear port may be adapted for receipt of the outflow adapter at a male port extending from one end of the main passage. As illustrated, the male port as illustrated may include an annular ring extending towards an annular taper. The terminus and the radial passage are generally positioned for fluidic communication with the trephine so that the outflow adapter may be used to facilitate connections to the vacuum source or irrigant sources with for example, the radial passage. In addition, the outflow adapter may facilitate receipt of various instruments or manual control of entering or exiting fluid from the surgical site with a rear aperture positioned opposite the male port and associated with the outflow adapter.

In an alternative embodiment, the present invention provides for an improved tip position determining assembly for visually determining the entry of the instrument tip into the internal surgical site. In this embodiment, a transparent endoscopic portal includes a proximate chamber housing the membrane and having a rear port extending from the rear of the chamber and an elongated portal cannula extending forwardly from the chamber towards the portal tip. The rear port is generally axially aligned with the portal cannula providing better fluid and air transmission characteristics.

The trephine extends from a trephine body towards the elongated trephine cannula, which in turn extends from a distal end of the trephine body towards a trephine cannula tip. In addition, an endoscopic side port extends radially outward from the chamber. The endoscopic side port, as illustrated is in communication with the chamber and is positioned on the distal side of the membrane which has at least one slit formed for passage of the trephine therethrough. The slit forms a plurality of slit openings for fluidic communication of the rear port with the chamber, the slit being resilient and configured for normally closed operation. In addition, the improved assembly includes a visual instrument received by the trephine, which is sealingly secured by the slit as the visual instrument extends along the elongated trephine cannula, providing images of a visual horizon associated with the exterior cylindrical sidewall surface of the endoscopic portal.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is a side elevational view of an embodiment of a portal scope for use in the present invention.

FIG. 2 is a side elevational view of an embodiment of a trephine instrument for use in the present invention.

FIG. 3 is a side elevational view of the trephine instrument inserted through the portal scope, with a fluid source connected to a side port of the trephine instrument and a vacuum source connected to a side port of the portal instrument.

FIG. 4 is an enlarged cross sectional view taken on line 4-4 of FIG. 1 and illustrates an embodiment of a membrane of the portal scope with a pair of slits crossed at a right angle.

FIG. 5 is an enlarged cross sectional view taken on line 5-5 of FIG. 3 and illustrates the portal membrane with the trephine cannula extending therethrough.

FIG. 6 is a greatly enlarged cross sectional view taken on line 6-6 of FIG. 3 and illustrates a coaxial relationship between the trephine cannula and the portal cannula and shows an annular passage therebetween.

FIGS. 7 and 8 are side elevational views of the trephine instrument being withdrawn from the portal scope.

FIG. 9 is a side elevational view similar to FIG. 3 and illustrates the trephine instrument inserted through an alternative configuration of a portal scope.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

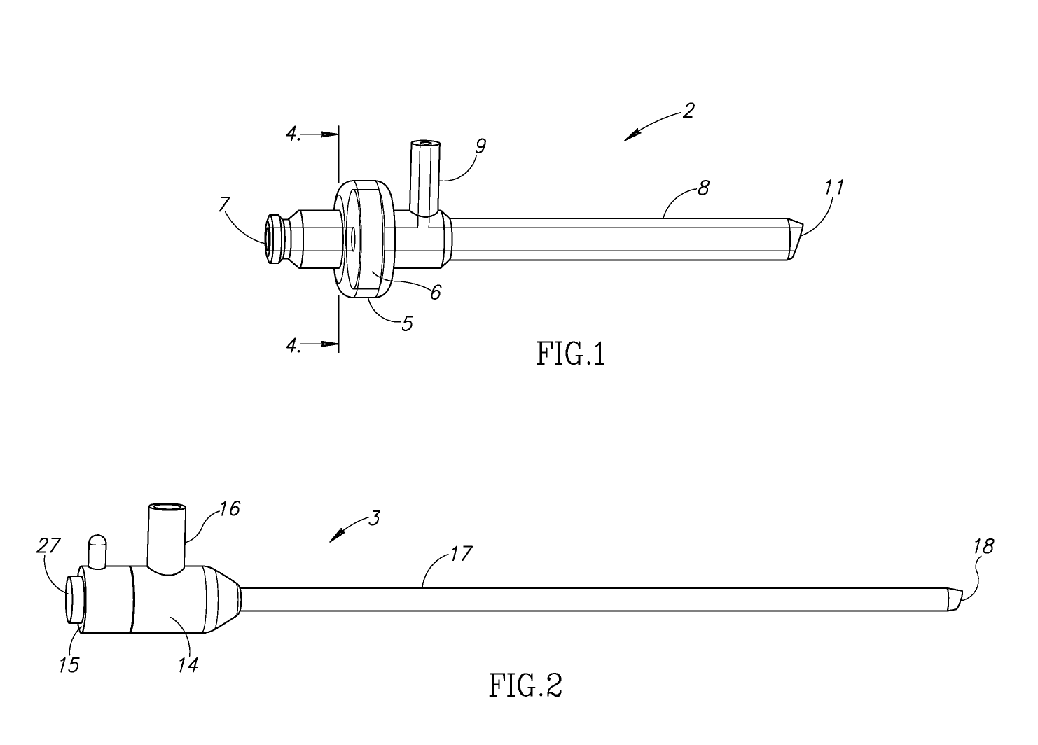

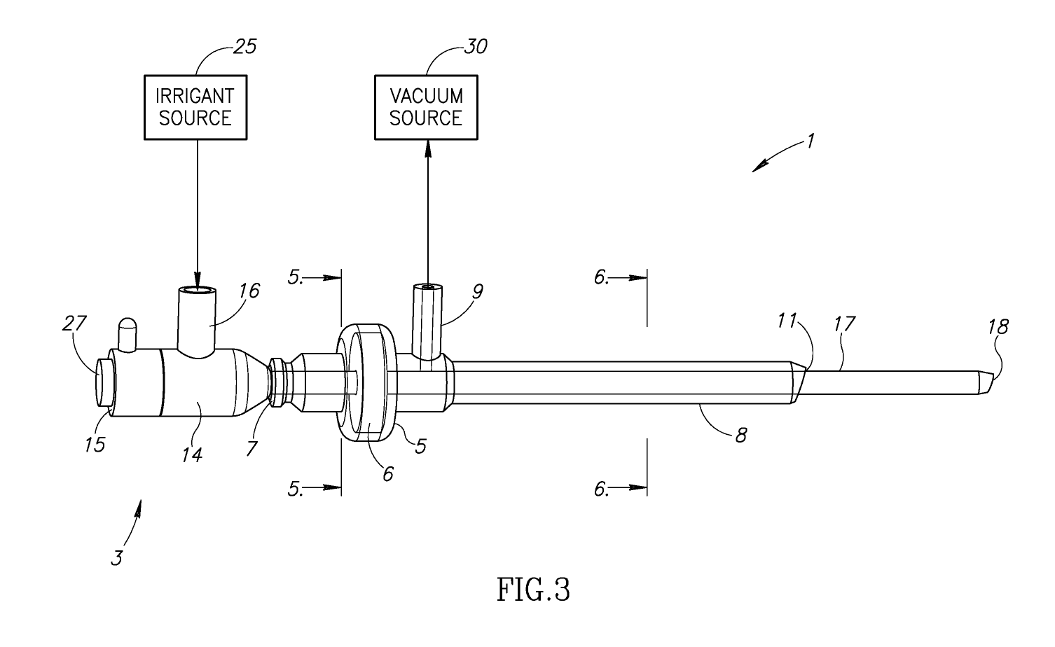

Referring to the drawings in more detail, the reference numeral 1 (FIGS. 3 and 7-9) generally designates an embodiment of a tip position determining assembly 1 of endoscopic instruments for determining fluidically the entry of a tip or tips of the instruments into an internal surgical site. Referring to FIGS. 1 and 2, the illustrated assembly 1 includes an endoscopic portal instrument or scope 2 (FIG. 1) and a trephine instrument or trephine 3 (FIG. 2). As illustrated in FIGS. 3 and 7-9, the trephine instrument 3 is inserted through the portal scope 2 to form the assembly 1.

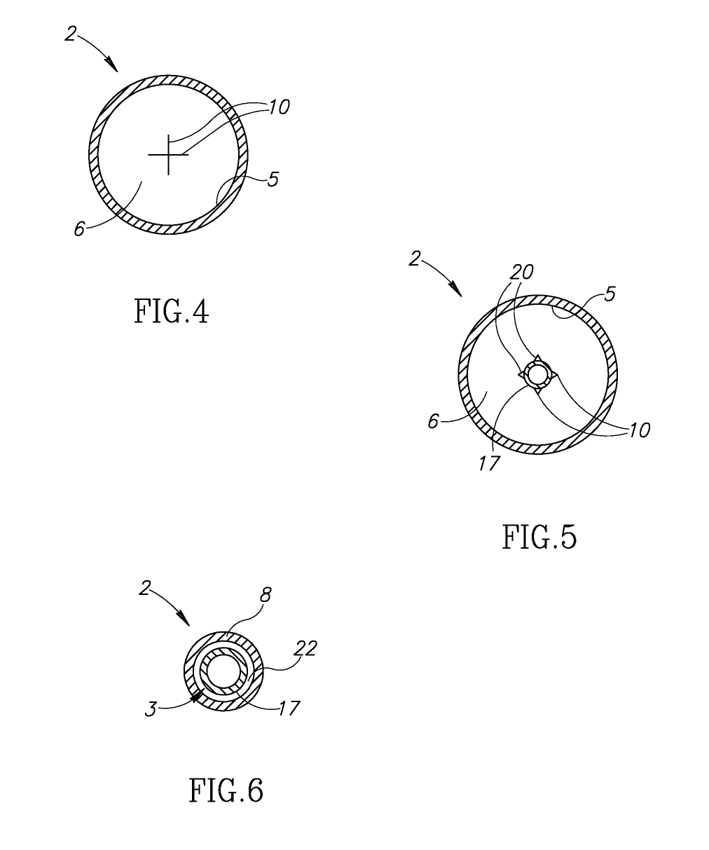

Referring to FIG. 1, the illustrated portal scope 2 includes an enlarged membrane chamber 5 having a membrane or diaphragm member 6 sealingly positioned therein. The membrane chamber 5 has a rear port 7 extending rearwardly therefrom and an elongated portal cannula 8 extending forwardly therefrom, the rear port 7 and the portal cannula 8 being axially aligned. A side port 9 extends radially from the chamber 5 and communicates with the chamber 5 on a distal side of the membrane 6. As shown in FIG. 4, the membrane 6 has at least one and preferably a crossed pair of slits 10 formed therethrough, as will be described below. However, it is foreseen that other slit patterns could be employed. The membrane 6 is formed of a resilient material such that the slits 10 are normally closed and form a partial seal between proximal and distal sides of the chamber 5. Thus, the side port 9 communicates directly with the portal cannula 8 through the distal side of the chamber, while the rear port 7 communicates with the cannula 8 through the slits 10 in the membrane 6. The portal cannula 8 terminates distally in a portal cannula tip 11. The illustrated tip 11 is conically tapered and cut off at an angle.

Referring to FIG. 2, the illustrated trephine instrument 3 is formed by a trephine body 14 having a proximal rear port 15 and a radial side port 16, both communicating with an internal trephine chamber (not shown). An elongated trephine cannula 17 extends from a distal end of the trephine body and terminates in a trephine cannula tip 18. The tip 18 may be conically tapered and cut off at an angle, although other tip configurations are within the scope of the present embodiment. The cannula 17 has an outer diameter which is less than an inner diameter of the portal cannula 8. In general, the trephine cannula 17 is longer than the entire length of the portal scope 2.

Referring to FIG. 3, the tip location determining assembly 1 is formed by insertion of the trephine cannula 17 through the entire length of the portal scope 2, with the trephine cannula 17 passing through the rear port 7, the slits 10 in the membrane 6, the membrane chamber 6, and the portal cannula 8. The rear port 7 of the portal scope 2 is brought close to the trephine body 14, but is spaced therefrom to avoid blockage of the rear port 7. As shown in FIG. 5, when the trephine cannula 17 passes through the slits 10 of the membrane 6, small roughly triangular openings 20 are formed at the ends of the slits 10. Thus, it is necessary for the slits 10 to have lengths greater than the outer diameter of the trephine cannula 17. The openings 20 enable some fluid communication between the rear port 7 and the membrane chamber 5, as will be described below. Because the inner diameter of the portal cannula 8 is greater than the outer diameter of the trephine cannula 17, an annular cannula gap 22 is formed between the overlapped sections of the portal cannula 8 and the trephine cannula 17, enabling fluid communication through the portal cannula 8 external to the trephine cannula 17.

In endoscopic surgery such as joint surgery, a guide wire (not shown) is often inserted through an external incision with the aid of radiant imaging, such as a fluoroscope (not shown). In joint surgery, the joint is typically surrounded by a joint or articular capsule which is pierced by insertion of the guide wire. The opening made by the guide wire is enlarged by the insertion of progressively larger diameter dilators until the desired opening is achieved. Although the present invention is described with particular application to arthroscopic surgery, it is not intended to be limited to such an application.

The assembly 1 is prepared for insertion toward the surgical site by connection of an irrigant source 25 to either the rear port 15 or the side port 16 of the trephine 3. The assembly 1 illustrated in FIG. 3 has the irrigant source 25 connected to the side port 16. A plug 27 is inserted in the unused port, in this case, the rear port 15. A vacuum source 30 is connected to the side port 9 of the portal scope 2. The assembly 1 is then telescoped over the guide wire, and the tip 18 of the trephine cannula 17 is inserted into the incision. The irrigant source 25 and the vacuum source 30 are activated as insertion continues with insertion of the tip 11 of the portal scope 2 into the incision. Suction from the vacuum source 30 is relieved to some degree by passing air into the membrane chamber 5 through the openings 20 at the ends of the membrane slits 10. The surgeon observes the portal cannula 8 and the side port 9 for changes in flow through the portal cannula 8 as the tips 18 and 11 are advanced toward the surgical site.

Along the way, the irrigant is issuing from the tip 18 of the trephine cannula 17. However, negative pressure may also occur as flow into the tip 11 of the portal cannula 11 is blocked by tissues or other obstructions encountered between the incision and the surgical site. Based upon the differential pressure between the vacuum source and the tip 18 or rear port 15, some air may pass from the rear port 15 to the vacuum source. Alternatively or additionally, some of the irrigant and other fluids such as blood may enter the portal tip 11 and become visible flowing through the portal cannula 8 toward the side port 9.

When the trephine tip 18 enters the surgical site, such as within a joint capsule, the irrigant pressurizes the capsule, such that when the portal tip 11 enters the site, the irrigant is drawn into the portal tip 11 in an appreciably increased quantity and flows back through the portal cannula 8 toward the side port 9. This increased flow is observable by the surgeon who then knows that the portal tip 11 has entered the surgical site or joint capsule.

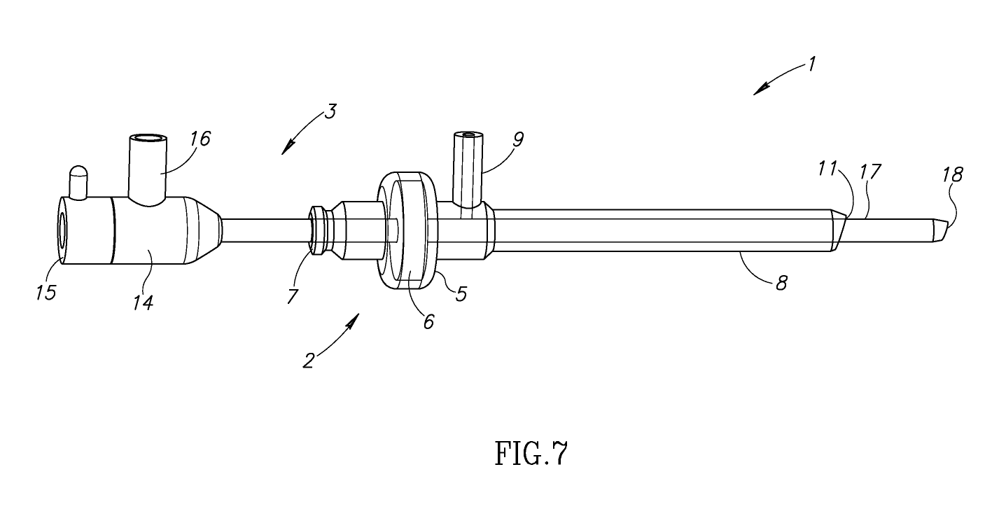

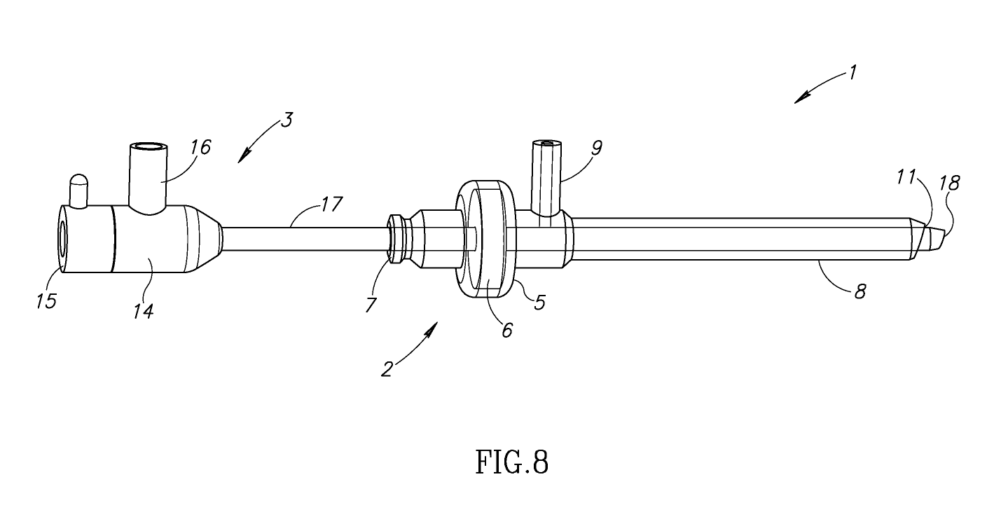

Once the portal tip 11 is positioned a short distance into the surgical site, the vacuum source 30 and irrigant source 25 can be deactivated, and the trephine 3 can be withdrawn from the surgical site and the portal scope 2, as is illustrated in FIGS. 7 and 8. The portal scope 2 is left in place to provide a path for the insertion of other endoscopic instruments or the exchange of fluids with the surgical site.

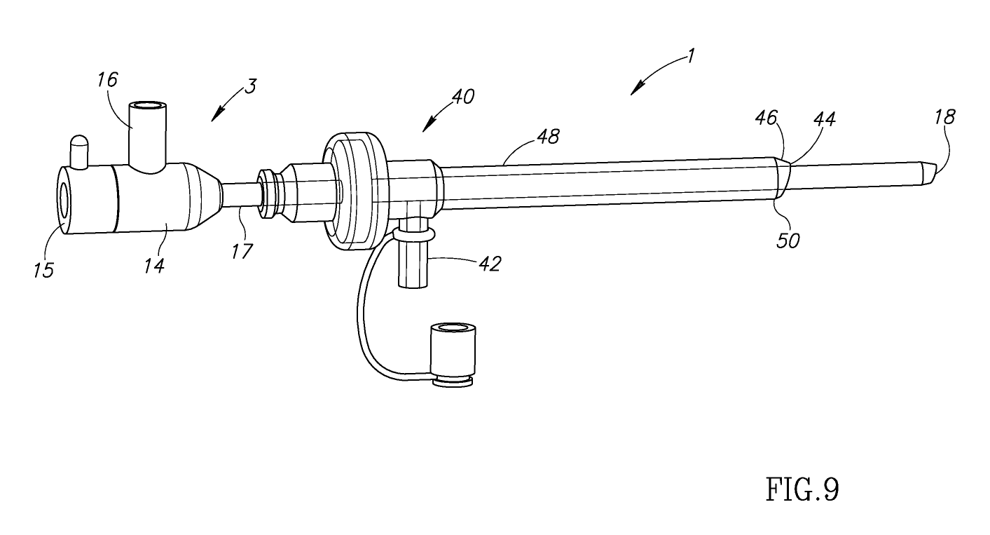

FIG. 9 illustrates the assembly 1 including the trephine instrument 3 inserted through an alternative configuration of a portal scope 40. The portal scope 40 differs from the portal scope 2 in that a side port 42 of the portal scope 40 is on an opposite side of a longitudinal axis thereof from an extreme end 44 of a tip section 46 of a portal cannula 48 of the scope 40. Additionally, the illustrated portal scope 40 includes a circumferential rim 50 at the tip section 46 which functions in a manner similar to a thread and helps retain the tip 46 of the scope 40 within a surgical site. The portal scope 40 is preferred for certain types of endoscopic surgeries, such as shoulder joint surgery. In other respects, the portal scope 40 is substantially similar to the portal scope 2, particularly with respect to use of the assembly 1 to fluidically determine entry of the tip section 46 into a surgical site.

In another alternative embodiment, the cannula tip 11 includes a lens or prism element associated with the distal end of the tip 11. The lens may include a planar surface associated with the inner diameter of the cannula 17 which may be conically configured for presenting an angled lens surface. A concave surface associated with the lens may extend radially inwardly from the inner cannula surface for broadening the field of view. As desired, the user may extend the trephine instrument 3 axially towards the rear port 7, allowing for greater visualization of the surrounding areas through the lens which presents the desired refraction index for magnifying the surrounding objects. The lens may be fabricated from a suitable optical plastic or resin material which are generally known to the art.

A removable outflow adapter 60 may be rearwardly associated with the trephine 3, the removable outflow adapter having a main passage 62 extending between a terminus 64 towards a male port 66 and a radial passage 68 extending radially outward from the main passage 62. A female connector 70 associated with the proximal rear port 72 may be adapted for receipt of the outflow adapter 60 at a male port 66 extending from one end of the main passage 62. As illustrated, the male port 66 as illustrated may include an annular ring 74 extending towards an annular taper 76. The terminus 64 and the radial passage 68 are generally positioned for fluidic communication with the trephine 3 so that the outflow adapter 60 may be used to facilitate connections to the vacuum source or irrigant sources with for example, the radial passage 68. In addition, the outflow adapter may facilitate receipt of various instruments or manual control of entering or exiting fluid from the surgical site with a rear aperture positioned opposite the male port 66 and associated with the outflow adapter 60. further information about an exemplary outflow adapter in general is disclosed in U.S. patent application Ser. Nos. 12/908,879 and 12/908,877 presently pending and filed Oct. 21, 2010 by applicant, the contents of which are incorporated herein by reference.

In an alternative embodiment, the present invention provides for an improved tip position determining assembly 101 for visually determining the entry of the instrument tip into the internal surgical site. In this embodiment, a transparent endoscopic portal 102 includes a proximate chamber 5 housing the membrane 6 and having a rear port 7 extending from the rear of the chamber and an elongated portal cannula 8 extending forwardly from the chamber 5 towards the portal tip 1. The rear port 7 is generally axially aligned with the portal cannula 8 providing better fluid and air transmission characteristics.

As illustrated, the trephine 3 extends from a trephine body 14 towards the elongated trephine cannula 17, which in turn extends from a distal end of the trephine body 14 towards a trephine cannula tip 18. In addition, an endoscopic side port 9 extends radially outward from the chamber 5. The endoscopic side port 9, as illustrated is in communication with the chamber 5 and is positioned on the distal side of the membrane 6 which has at least one slit 10 formed for passage of the trephine therethrough. The slit 10 forms a plurality of slit openings 20 for fluidic communication of the rear port 7 with the chamber 5. In general, the slit 10 is fabricated from a resilient material for normally closed operation, however, other materials may be used in accordance with the present invention.

A visual instrument 52 is received by the trephine in the improved assembly 101. The visual instrument 52 may include a number of visual surgical instruments, including but not limited to those commercially available video endoscope instruments. The visual surgical instrument 52 is generally sealingly secured by the slit 10 as the visual instrument 52 extends along the elongated trephine cannula 17, providing images of a visual horizon 54 associated with the exterior cylindrical sidewall surface 12 of the endoscopic portal 2.

In general the visual horizon 54 varies in relation to the distance between the tip of the visual instrument 52 and the trephine cannula tip 18. As the tip of the visual instrument 52 moves closer to the trephine cannula tip 18 towards the surgical site, the visual horizon 52 increases radially according to known geometric formulas, depending on the geometry of the trephine cannula 17. Additionally, the visual horizon 54 provides for visual observation of additional surgical instruments or other objects near the exterior portal surface on the way within the visual horizon 54 along towards the surgical site. Optionally, an alignment axis 56 associated with the exterior surface 12 of the portal cannula 8 may extend along the exterior surface 12, parallel to a longitudinal axis 58 associated with the portal cannula 8. The alignment axis 56 allows for alignment of various instruments within the visual horizon 54 as they extend towards the surgical site. In another alternative embodiment, a plurality of apertures 80 are circumferentially spaced along the cannula tip 11. These apertures will help remove unwanted debris and may be helpful as the visual instrument 54 travels towards the surgical site.

It is to be understood that while certain forms of the present invention have been illustrated and described herein, it is not to be limited to the specific forms or arrangement of parts described and shown.

What is claimed and desired to be secured by Letters Patent is:

1. A tip position determining assembly for fluidically determining the entry of an instrument tip into an internal surgical site, the assembly comprising:
an endoscopic portal having a proximately positioned chamber having a membrane sealingly positioned therein;
said chamber having a rear port rearwardly extending therefrom and an elongated portal cannula extending forwardly from said chamber towards a portal tip, the rear port and the portal cannula being axially aligned;
a trephine extending from a trephine body towards an elongated trephine cannula;
an endoscopic side port extending radially from said chamber and in communication therewith on a distal side of the membrane, said membrane having at least one slit formed for passage of said trephine therethrough;
a plurality of slit openings associated with said slit for fluidic communication of said rear port with said chamber; and
an irrigant source connectably secured to said trephine body and a vacuum source connectably secured to said endoscopic portal whereby said irrigant is observed passing through said member along said trephine cannula and into said portal tip as said instrument tip enters said surgical site.

2. The tip position determining assembly of claim 1 further comprising a cannula gap formed between said portal cannula and said trephine cannula for fluidic communication through said portal cannula.

3. The tip position determining assembly of claim 1 wherein said membrane is resilient.

4. The tip position determining assembly of claim 1 wherein a proximal side of said membrane is at least partially sealed from the distal side by said slits wherein said slits are normally closed.

5. The tip position determining assembly of claim 1 wherein each of said slits has a length greater than the outer diameter of said trephine cannula.

6. The tip position determining assembly of claim 1 wherein said vacuum source is in communication with said membrane chamber through said slit openings.

7. The tip position determining assembly of claim 1 wherein said elongated trephine cannula extends from a distal end of said trephine body towards a trephine cannula tip, said trephine cannula tip being spaced from said portal tip presenting an annular cannula gap therebetween.

8. The tip position determining assembly of claim 7 wherein said trephine cannula tip is frustroconical.

9. The tip position determining assembly of claim 1 wherein said trephine cannula is longer than the endoscopic portal.

10. The tip position determining assembly of claim 1 wherein said trephine body comprises a radial side port extending radially outward and a proximal rear port extending rearwardly therefrom one of said rear and side ports being adapted for connectably receiving said irrigant source.

11. The tip position determining assembly of claim 1 wherein said endoscopic portal further includes a tip section associated with said portal tip and presenting an angled lens surface.

12. The tip position determining assembly of claim 1 wherein said endoscopic portal further includes a circumferential rim at a tip section associated with said portal tip.

13. The tip position determining assembly of claim 10 further comprising:
a removable outflow adapter having a main passage extending between a terminus towards a male port and a radial passage extending radially outward therefrom;
said outflow adapter resealeably received by a female connector associated with said proximal rear port;
said male port extending from an annular ring towards an annular taper adapted for receipt by said female connector;
said terminus and said radial passage in fluidic communication with said trephine, one of said terminus and radial passage operably connected to one of said vacuum and irrigant source.

14. An improved tip position determining assembly for determining the entry of an instrument tip into an internal surgical site, the assembly comprising:
a transparent endoscopic portal having a proximately positioned chamber having a membrane sealingly positioned therein;
said chamber having a rear port rearwardly extending therefrom and an elongated portal cannula extending forwardly from said chamber towards a portal tip, the rear port and the portal cannula being axially aligned;
a trephine extending from a trephine body towards an elongated trephine cannula;
said elongated trephine cannula extending from a distal end of said trephine body towards a trephine cannula tip;
an endoscopic side port extending radially from said chamber and in communication therewith on a distal side of the membrane, said membrane having at least one slit formed for passage of said trephine therethrough;
a plurality of slit openings associated with said slit for fluidic communication of said rear port with said chamber; and
a visual instrument received by said trephine and sealingly secured by said slit as said visual instrument travels along said elongated trephine cannula providing images of a visual horizon associated with an exterior surface of said endoscopic portal.

15. The improved tip position determining assembly according to claim 14 wherein said visual horizon varies in relation to the distance between said visual instrument and said trephine cannula tip as the visual instrument travels along said elongated trephine cannula towards the surgical site.

16. The improved tip position determining assembly according to claim 14 wherein said visual horizon further includes a tip of a surgical instrument passing near said exterior surface towards the surgical site.

17. The improved tip position determining assembly according to claim 14 further comprising an alignment axis extending along said exterior surface and parallel to a longitudinal axis associated with the portal cannula, said alignment axis being located within said visual horizon for aligning said instrument tip.

18. The tip position determining assembly of claim 14 further comprising:
a removable outflow adapter having a main passage extending between a terminus towards a male port and a radial passage extending radially outward therefrom;
said outflow adapter resealeably received by a female connector associated with a proximal rear port extending rearwardly from said trephine body;
said male port extending from an annular ring towards an annular taper adapted for receipt by said female connector; and
said terminus and said radial passage in fluidic communication with said trephine, one of said terminus and radial passage operably connected to one of said vacuum and irrigant source.

19. The nontransparent scope of claim 14 further comprising:
a main body presenting cannula extending towards said surgical site for passing the surgical instrument therethrough;
said cannula having a proximal end and a distal end associated with a terminal point;
said portal adapted for slidable receipt of said cannula;
a removable outflow adapter associated with said main body and extending rearwardly therefrom;
a male port associated with said outflow adapter and including an annular ring enclosing a main passage and an annular taper extending from said annular ring;
a proximate rear port associated with said main passage and adapted for receiving said male port for communication between said main passage and said cannula;
a radial passage associated with said outflow adapter and radially extending therefrom, said radial passage in communication with said main passage.

20. The tip position determining assembly of claim 14 wherein said endoscopic portal further includes a tip section associated with said portal tip and presenting a plurality of apertures circumferentially spaced around said portal tip.
